(19) United States
(12) Patent Application Publication
(10) Pub. No.: US 2010/0179663 A1
(43) Pub. Date: Jul. 15, 2010

(54) ACETABULAR PROSTHETIC DEVICES

(75) Inventor: Amiram Steinberg, Avihail (IL)

Correspondence Address: HAYNES AND BOONE, LLP;IP Section, 2323 Victory Avenue, Suite 700, Dallas, TX, 75219, US

(73) Assignee: Active Implants Corporation, Memphis, TN (US)

(21) Appl. No.: 12/597,973

(22) PCT Filed: May 21, 2008
(86) PCT No.: PCT/US08/64352

§ 371 Date: Mar. 3, 2010

Related U.S. Application Data
(60) Provisional application No. 60/939,327, filed on May 21, 2007.
(60) Provisional application No. 60/946,522, filed on Jun. 27, 2007.

(51) Int. Cl. A61F 2/34

(52) U.S. Cl. 623/22.24

(57) ABSTRACT

Devices, apparatus, and systems for replacing at least some of the functionality of the natural hip joint and associated methods of implantation are disclosed. In one aspect a prosthetic acetabular cup system is provided. The system includes a metal shell comprising an outer surface for securely engaging a prepared portion of an acetabulum, an opposing inner surface, and at least one snap-fit engagement feature associated with the inner surface. The metal shell has a thickness less than about 1.0 mm between the outer surface and the inner surface. The system also includes a pliable articulating component having an outer surface including at least one snap-fit engagement feature sized and shaped to snap-fittingly engage the at least one snap-fit engagement feature of the metal shell. The pliable articulating component also includes an inner surface for articulatingly receiving a femoral head. The second component has a thickness less than about 3.0 mm.

### PRIORITY

This application is a United States national phase application of co-pending international patent application number PCT/US2008/064352, filed May 21, 2008, which claims priority to U.S. Provisional patent application No. 60/946,522, filed Jun. 27, 2007 and U.S. Provisional patent application No. 60/939,327, filed May 21, 2007, the disclosures of which are incorporated herein by reference.

### TECHNICAL FIELD

Embodiments of the present disclosure relate generally to medical prosthetic devices, including prosthetic hip joint components, and associated methods of implantation and treatment.

### BACKGROUND

The present disclosure relates to devices, apparatus, and systems for replacing at least some of the functionality of the natural hip joint and methods of implanting such devices, apparatus, and systems. The natural hip joint is a ball-and-socket joint formed by the articulating interaction of the rounded head of the femur with the acetabulum of the pelvis. The articulating surfaces of both the head of the femur and the acetabulum are covered with articular cartilage. Various conditions can cause damage to the hip joint resulting in debilitating pain, arthritis, and/or limited mobility. In some instances, hip arthroplasty has been used to treat such conditions.

Although existing devices and methods associated with prosthetic hip joint components have been adequate in some respects, they have not been satisfactory in all respects. The present disclosure overcomes one or more of the shortcomings of the existing devices and methods.

### SUMMARY

In one embodiment, a prosthetic device for positioning within a hip joint is disclosed.

In some instances, the prosthetic device is comprised of two components: a thin metal shell and a pliable articulating component. In some embodiments, the pliable articulating component is made of a pliable structural material validated for medical devices used in vivo and having mechanical properties characterized by a non-linear stress-strain relationship defined by a polynomial mathematical curve having more than two coefficients, such as the Mooney Rivlin coefficients. In some instances, the material has strength and/or elasticity substantially similar to human cartilage. In some instances, the material is a polyurethane based material, which may be polycarbonate polyurethane in some embodiments, and may be a Bionate Polyurethane in some embodiments. The material of the metal shell and the pliable articulating component operate together to dissipate bone stress and strain associated with the hip joint during the life of the device to help preserve and/or regenerate bone strength.

In another embodiment, a prosthetic device for implantation into a hip joint is disclosed. The prosthetic device includes a first component comprising an outer surface for securely engaging a bony portion of an acetabulum and an opposing inner surface for receiving a second component. The outer surface has a generally semi-spherical profile. The inner surface also has a generally semi-spherical profile concentric with the outer surface. The first component has a substantially uniform thickness less than about 1.0 mm between the outer surface and the inner surface. The first component also includes at least one annular protrusion extending circumferentially from the inner surface. The at least one annular protrusion has a height less than about 1.0 mm relative to the inner surface. The first component comprises a substantially rigid first material. The prosthetic device also includes a second component. The second component includes an outer surface having a generally semi-spherical profile for engagement with the inner surface of the first component. In that regard, the outer surface includes at least one annular recess extending circumferentially into the outer surface with a depth less than about 1.0 mm. The at least one annular recess is shaped to receive the at least one annular protrusion of the first component. The second component also includes an inner surface for mating with a femoral head. The inner surface has a generally semi-spherical profile concentric with the outer surface. The second component has a substantially uniform thickness less than about 3.0 mm between the outer surface and the inner surface. The second component comprising a pliable second material. In some instances, the first and second components have a total thickness between the outer surface of the first component and the inner surface of the second component that is approximately equal to a thickness of a native articular cartilage of the acetabulum adjacent the bony portion of the acetabulum. In some instances, the prosthetic device also includes a prosthetic femoral head for mating with the inner surface of the second component.

In another embodiment, a prosthetic acetabular cup system is disclosed. The system includes a metal shell comprising an outer surface for securely engaging a prepared portion of an acetabulum, an opposing inner surface for receiving a pliable articulating component, and at least one snap-fit engagement feature associated with the inner surface. The metal shell has a thickness less than about 1.0 mm between the outer surface and the inner surface. The system also includes a pliable articulating component. The pliable articulating component includes an outer surface for engaging the inner surface of the metal shell, including at least one snap-fit engagement feature associated sized and shaped to snap-fittingly engage the at least one snap-fit engagement feature of the metal shell. The pliable articulating component also includes an inner surface for receiving a femoral head. The pliable articulating component has a thickness less than about 3.0 mm between the outer surface and the inner surface. In some instances, the at least one snap-fit engagement feature of the metal shell comprises at least one recess and the at least one snap-fit engagement feature of the pliable articulating component comprises at least one projection. In some instances, the at least one snap-fit engagement feature of the metal shell comprises at least one projection and the at least one snap-fit engagement feature of the pliable articulating component comprises at least one recess. In some instances, the metal shell and the pliable articulating component have a total thickness between the outer surface of the metal shell and the inner surface of the pliable articulating component that is approximately equal to a thickness of a native articular cartilage of the acetabulum.

In another embodiment, a method of implanting an artificial acetabular component is disclosed. The method includes removing at least a portion of an articular cartilage of an acetabulum to access a bony portion of the acetabulum and engaging an outer surface of a metal shell with the bony portion of the acetabulum, where the metal shell has an opposing inner surface and a thickness of less than about 1.0 mm between the outer surface and the inner surface. The method also includes snap-fitting a pliable articulating component into engagement with the inner surface of the metal shell, where the pliable articulating component has a thickness less than about 3.0 mm between an outer engagement surface and an inner articulating surface such that when snap-fittingly engaged the metal shell and the pliable articulating component have a total thickness between the outer surface of the metal shell and the inner articulating surface of the pliable articulating component that is approximately equal to a thickness of the removed articular. In some instances, snap-fitting the pliable articulating component into engagement with the inner surface of the metal shell comprises snap-fitting at least one projection extending from the inner surface of the metal shell with at least one recess extending into the outer engagement surface of the pliable articulating component. In some instances, the method also includes wetting at least the outer engagement surface of the pliable articulating component prior to snap-fitting the pliable articulating component into engagement with the metal shell.

### BRIEF DESCRIPTION OF DRAWINGS

Other features and advantages of the present disclosure will become apparent in the following detailed description of embodiments of the disclosure with reference to the accompanying of drawings, of which:

FIG. 1 is a diagrammatic cross-sectional view of an arrangement incorporating a prosthetic device according one embodiment of the present disclosure.

FIG. 2 is a diagrammatic perspective view of a component of the prosthetic device of FIG. 1 according to one embodiment of the present disclosure.

FIG. 3 is a diagrammatic cross-sectional side view of the component of FIG. 2.

FIG. 4 is a diagrammatic bottom view of the component of FIGS. 2 and 3.

FIG. 5 is a diagrammatic cross-sectional view of another component of the prosthetic device of FIG. 1 according to one embodiment of the present disclosure.

FIG. 6 is a diagrammatic cross-sectional view of the component of FIGS. 2-4 assembled with the component of FIG. 5 to form the prosthetic device of FIG. 1.

FIG. 7 is a diagrammatic cross-sectional view of a prosthetic device similar to FIG. 6, but showing an alternative embodiment of the present disclosure.

FIG. 8 is a diagrammatic cross-sectional view of a prosthetic device similar to FIGS. 6 and 7, but showing another alternative embodiment of the present disclosure.

FIG. 9 is a diagrammatic cross-sectional view of a patient's unprepared acetabulum according to one aspect of the present disclosure.

FIG. 10 is a diagrammatic cross-sectional view of a patient's prepared acetabulum according to one aspect of the present disclosure.

FIG. 11 is a diagrammatic cross-sectional view of the component of FIGS. 2-4 implanted into the patient's prepared acetabulum according to one aspect of the present disclosure.

FIG. 12 is a diagrammatic cross-sectional view of the component of FIG. 5 assembled with the component of FIGS. 2-4 implanted into the patient's prepared acetabulum to form the prosthetic device of FIG. 1 according to one aspect of the present disclosure.

FIG. 13 is a diagrammatic cross-sectional view of the assembled prosthetic device implanted into the patient's prepared acetabulum as shown in FIG. 12 mated with a femoral head according to one aspect of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications in the described devices, instruments, methods, and any further application of the principles of the disclosure as described herein are contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure.

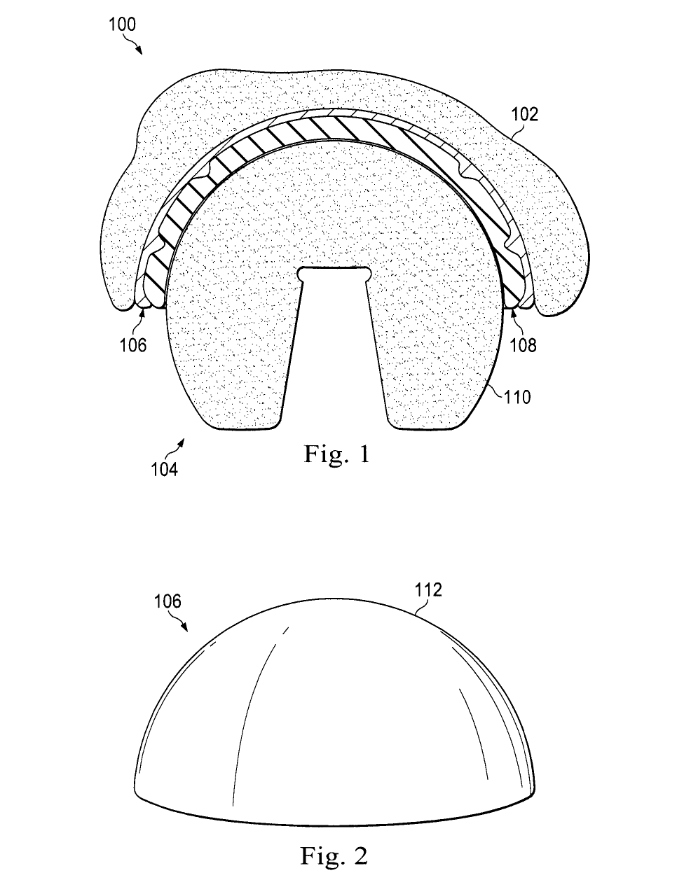

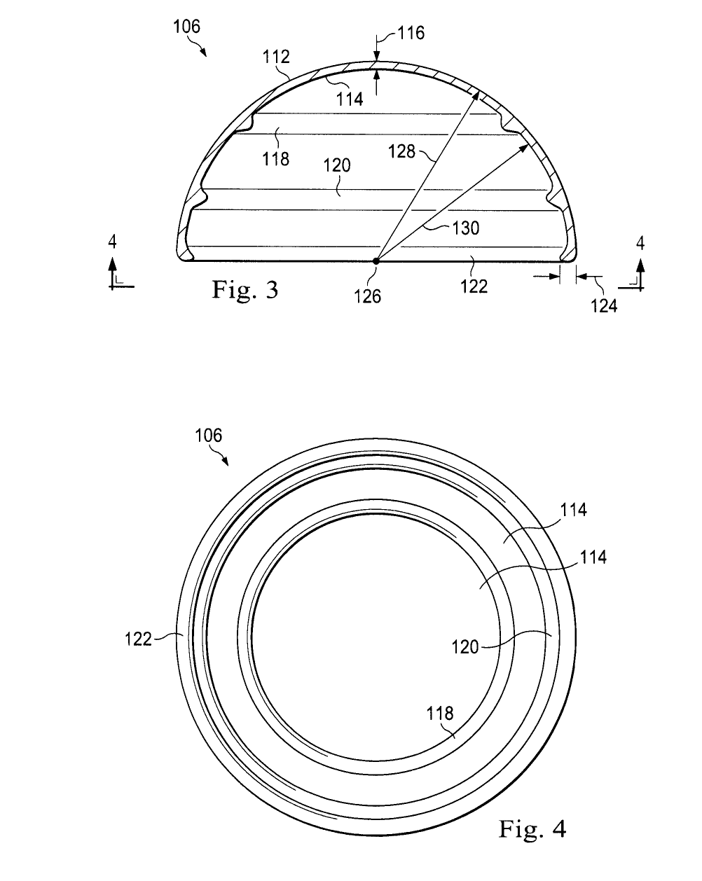

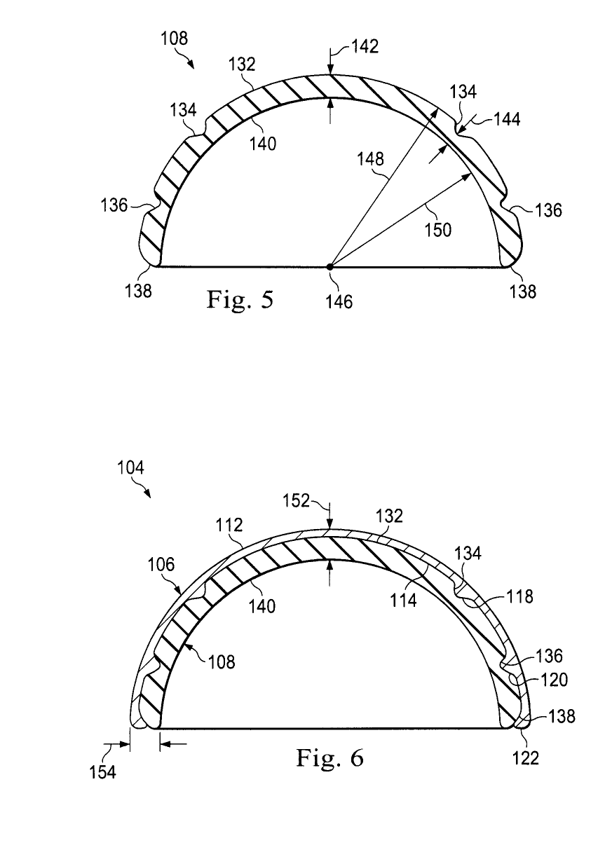

Referring now to FIGS. 1, 2, 3, 4, 5, and 6, shown therein is an arrangement 100 incorporating aspects of the present disclosure. The arrangement 100 includes an acetabulum 102 and a prosthetic system 104. The prosthetic system 104 includes at least a shell 106 and an articulating component 108. A femoral head 110 is also shown. The femoral head 110 is a prosthetic component in some instances such that the femoral head 110 is considered part of the prosthetic system 104. In other instances, the femoral head 110 is a natural femoral head and is not considered part of the prosthetic system 104. FIG. 1 is a diagrammatic cross-sectional view of the arrangement 100; FIG. 2 is a diagrammatic perspective view of the shell 106 of the prosthetic system 104 according to one embodiment of the present disclosure; FIG. 3 is a diagrammatic cross-sectional side view of the shell 106; FIG. 4 is a diagrammatic bottom view of the shell 106; FIG. 5 is a diagrammatic cross-sectional view of the articulating component 108 of the prosthetic system 104 according to one embodiment of the present disclosure; and FIG. 6 is a diagrammatic cross-sectional view of the shell 106 assembled with the articulating component 108.

Referring more specifically to FIGS. 2-4, the shell 106 includes an outer surface 112 for engaging a prepared portion of the acetabulum 102. In that regard, in some instances, the outer surface 112 of the shell 106 is treated to enhance engagement between the outer surface and the acetabulum 102. In some instances, the outer surface 112 is roughened to increase the friction between the acetabulum and the device. Further, the engagement surface may be treated with biologics to encourage ingrowth of bone and/or articular cartilage. In some instances, the engagement surface receives one or more surface treatments as described in U.S. patent application Ser. No. 10/497,897 titled “CUSHION BEARING IMPLANTS FOR LOAD BEARING APPLICATIONS,” hereby incorporated by reference in its entirety. Further, in some instances the outer surface 112 includes structural features (not shown) for encouraging engagement between the shell 106 and the acetabulum 102. For example, the outer surface 112 includes projections, recesses, and/or combinations thereof in some instances. Generally, the outer surface 112 may be rigidly secured to the acetabulum in any medically suitable manner. In that regard, in some instances the outer surface 112 will engage a bony portion of the acetabulum 102. In some instances, the outer surface 112 will engage at least some articular cartilage of the acetabulum 102.

Referring more specifically to FIGS. 3 and 4, the shell 106 includes an inner surface 114 opposite the outer surface 112. In the present embodiment, the outer surface 112 is substantially semi-spherical. Similarly, the inner surface 114 is also substantially semi-spherical and concentric relative to the outer surface 112. The outer surface 112 and the inner surface 114 are separated by a thickness 116 of the shell 106. Generally, the thickness 116 is less than about 1.0 mm and, in some embodiments, is less than about 0.5 mm. In some embodiments, the thickness is between about 0.5 mm and about 1.0 mm. In the present embodiment, three continuous annular protrusions 118, 120, and 122 extend circumferentially about the inner surface 114 of the shell. In that regard, the protrusion 122 at least partially defines the rim or boundary of the shell 106 as shown. The protrusions 118 and 120 are spaced from one another and positioned between the protrusion 122 and an apex of the inner surface 114. In the present embodiment, the protrusions 118, 120, 122 each have substantially similar rounded profiles such that the shell has a thickness 124 between the outer surface 112 and the apex of each of the protrusions. In that regard, the thickness 124 is generally less than about 2.0 mm and, in some embodiments, is less than about 0.5 mm. As discussed below, however, in other embodiments the protrusions 118, 120, 122 may have various other profiles instead of the illustrated rounded profiles. The protrusions 118, 120, 122 increase the rigidity, stiffness, and/or structural integrity of the shell 106. In that regard, the protrusions 118, 120, 122 serve to limit the flexing and deformation of the shell 106 in some instances. In some embodiments, the protrusions 118, 120, 122 provide a hoop strength to the shell 106 that limits deformation. Depending on the particular material utilized for the shell 106, additional or fewer protrusions and/or other stiffening structures may be utilized to obtain a desired stiffness for the shell.

From a center point 126, the substantially semi-spherical outer surface 112 is defined by a radius of curvature 128. Similarly, the substantially semi-spherical inner surface 116 is defined by a radius of curvature 130. The radius of curvature 128 is generally between about 24.0 mm and about 30.0 mm, but in some instances may be larger or smaller. Accordingly, the radius of curvature 130 is generally between about 23.0 mm and about 29.5 mm, but also may be larger or smaller in some instances.

Referring now to FIG. 5, the articulating component 108 includes an outer surface 132 shaped to mate with the inner surface 114 and protrusions 118, 120, 122 of the shell 106. In that regard, the outer surface 132 includes a pair of recesses 134, 136 sized and shaped to receive and engage the protrusions 118, 120, respectively. In that regard, the recesses 134, 136 have rounded profiles that are substantially the inverse of the profile of the protrusions 118, 120 of the shell 106. Generally, the engagement between the recesses 134, 136 and the protrusions 118, 120 anchors the articulating component 108 within the shell 106 and constrains movement of the articulating component relative to the shell. In some instances, the recesses 134, 136 and the protrusions 118, 120 snap-fit together. Further, the outer surface 132 includes a taper 138 adjacent its outer edge or rim. In that regard, the taper 138 is sized and shaped to mate with the protrusion 122 of the shell 106. In some instances, the taper 138 and the protrusion 122 snap-fit together similar to the recesses 134, 136 and the protrusions 118, 120. In some instances, the articulating component 108 includes deformation control elements or reinforced material adjacent to and/or defining the recesses 134, 136. In that regard, the deformation control elements and/or the reinforced material can strengthen the structural integrity of the articulating element 108 to prevent unwanted interruption to the inner articulating surface 140 that may be caused by abnormally heavy loading of the hip joint distributed through the projections 118, 120 into the articulating element 108.

In some embodiments, the articulating component 108 does not include recesses 134, 136 and the shell 106 does not include protrusions 118, 120, such that the articulating component 108 is anchored to the shell 106 via engagement of the protrusion or rim 122 with the taper or rim 138 of the articulating component. In that regard, the diameter of the opening defined by the protrusion or rim 122 is less than a maximum diameter of the articulating component 108, such that the articulating component is held within the shell 106 by the protrusion 122. In such embodiments, the articulating component 108 may have a substantially smooth outer surface for interfacing the with the shell 106. In one such embodiment, the articulating component 108 may also be anchored to the shell 106 via a liquid adhesion bond. In such instances, the liquid adhesion bond prevents separation of the articulating component 108 from the shell 106 while the protrusion 122 limits rotation of the articulating component 108 relative to the shell 106.

The articulating component 108 also includes an inner surface 140 opposite the outer surface 132. In the present embodiment, the outer surface 132 is substantially semi-spherical. Similarly, the inner surface 140 is also substantially semi-spherical and concentric relative to the outer surface 132. The outer surface 132 and the inner surface 140 are separated by a thickness 142 of the articulating component 108. Generally, the thickness 142 is between about 6.0 mm and about 1.5 mm. In some embodiments, the thickness 142 is less than about 3.0 mm and, in some further embodiments, is about 1.5 mm. The articulating component 108 also has a thickness 144 between the inner surface 140 and the trough or lowest point of each of the recesses 134, 136. In that regard, the thickness 144 is generally between about 5.0 mm and about 0.5 mm. In some embodiments, the thickness 144 is less than about 2.0 mm. As discussed below, however, in other embodiments the protrusions 118, 120, 122 may have various other profiles instead of the illustrated rounded profiles.

From a center point 146, the substantially semi-spherical outer surface 132 is defined by a radius of curvature 148. Similarly, the substantially semi-spherical inner surface 140 is defined by a radius of curvature 150 extending from the center portion 146. The radius of curvature 148 is generally between about 23.0 mm and about 29.5 mm, but in some instances may be larger or smaller. Accordingly, the radius of curvature 150 is generally between about 17.0 mm and about 28.5 mm, but also may be larger or smaller in some instances.

Referring now to FIG. 6, the shell 106 and the articulating component 108 are securely engaged with one another. In the present embodiment, the articulating component 108 is snap-fit within the shell 106. In that regard, the protrusions 118, 120 of the shell 106 snap-fit into the recesses 134, 136 in the articulating component 108. Further, the protrusion 122 of the shell 106 engages the taper 138 of the articulating component 108. In some instances, the interface between the protrusion 122 and the taper 138 provides a boundary to prevent unwanted or accidental disengagement of the articulating component 108 from the shell 106. In that regard, in some embodiments the protrusion 122 extends more prominently over the outer portion or rim of the articulating component 108 than illustrated in FIG. 6. In one such embodiment, the protrusion 122 secures the articulating component 108 to the shell 106 and the shell does not include protrusions 118 and 120. In some instances, engagement of the protrusions 118, 120, 122 with the articulating component 108 causes the articulating component 108 to deform as it is positioned within the shell 106. Specifically, portions of the articulating component, including its outer and inner surfaces are deformed inwardly such that the outer and inner surface are not substantially partially spherical. Instead, the outer and inner surfaces become partially elliptical or oblonged in some instances. For example, in some instances the apex of the outer surface of the articulating component 108 is positioned closer to the inner surface of the shell 106, than remaining portions of the outer surface until the protrusions 118, 120, 122 engage the recesses 134, 136 and taper 138, respectively.

When assembled, the shell 106 and the articulating component 108 have a combined thickness 152 adjacent the apex of the components between the outer surface 112 of the shell 106 and the inner surface 140 of the articulating member 108. When assembled, the shell 106 and the articulating component 108 also have a combined thickness 154 adjacent the rim of the components between the outer surface 112 of the shell 106 and the inner surface 140 of the articulating member 108. In the present embodiment, the thicknesses 152, 154 are substantially equal such that when assembled the shell 106 and articulating component 108 define a substantially constant thickness between the outer surface 112 and the inner surface 140. In that regard, the mating of the protrusions 118, 120 with the recesses 134, 136 and the mating of the protrusion 122 with the taper 138 results in the constant thickness. In other embodiments, the combined thickness extending between the outer surface 112 and the inner surface 140 is not substantially constant. In one particular embodiment, the thickness 154 adjacent the rim of the components is larger than the thickness 152 adjacent the apex of the components. In that regard, an increased thickness 154 adjacent the rim of the components is utilized to at least partially retain a femoral head within the articulating component in some instances.

In some instances, the combined thicknesses 152, 154 are substantially equal to or less than a thickness of the native cartilage of the acetabulum in some instances. In that regard, in some instances the combined thicknesses 152, 154 are substantially equal to or less than a desired or normal amount of articular cartilage, but not necessarily less than the articular cartilage of a particular patient. For example, in the case where a patient's articular cartilage has been damaged or removed, the thicknesses 152,154 may be greater than the thickness of the patient's articular cartilage. In some instances the combined thicknesses 152, 154 are substantially equal to or less than the actual articular cartilage of the patient. In that regard, in some instances the shell 106 and articulating component 108 are sized to substantially replace the space created by removal of articular cartilage and/or bone from the acetabulum prior to insertion of the shell 106 and articulating component 108. In some instances the shell 106 and articulating component 108 are sized to replace less than the space created by removing articular cartilage and/or bone from the acetabulum prior to insertion of the shell 106 and articulating component 108, such that a femoral head larger than the natural femoral head may be utilized.

While the shell 106 is shown as having protrusions 118, 120, and 122, in other embodiments the shell may have other engagement features for mating the with the articulating component 108. Similarly, while the articulating component 108 is shown as having recesses 134, 136 and taper 138, in other embodiments the articulating component may have other engagement features for mating with the shell. In that regard, each of the shell 106 and articulating component 108 may include projections, recesses, and combinations thereof sized and shaped to engage corresponding projections, recesses, and combinations thereof of the other component. In some instances the engagement features are similar to the engagement features of one or more of the prosthetic devices described in U.S. patent application Ser. No. 10/289,126 titled “ONE PIECE SNAP FIT ACETABULAR CUP,” U.S. patent application Ser. No. 10/497,897 titled “CUSHION BEARING IMPLANTS FOR LOAD BEARING APPLICATIONS,” U.S. patent application Ser. No. 10/515,486 titled “IMPLANTS,” U.S. patent application Ser. No. 11/688,153 titled “CERAMIC-ON-CERAMIC PROSTHETIC DEVICE COUPLED TO A FLEXIBLE BONE INTERFACE,” or PCT Application No. PCT/IL2006/000343 titled “IMPLANT DEVICES” (published as WO 2006/097932), each incorporated by reference in its entirety. It is recognized that the various combinations of projections and recesses described as being formed in the acetabulum by these references can instead by formed in one of the shell 106 and/or articulating component 108 in accordance with the present disclosure.

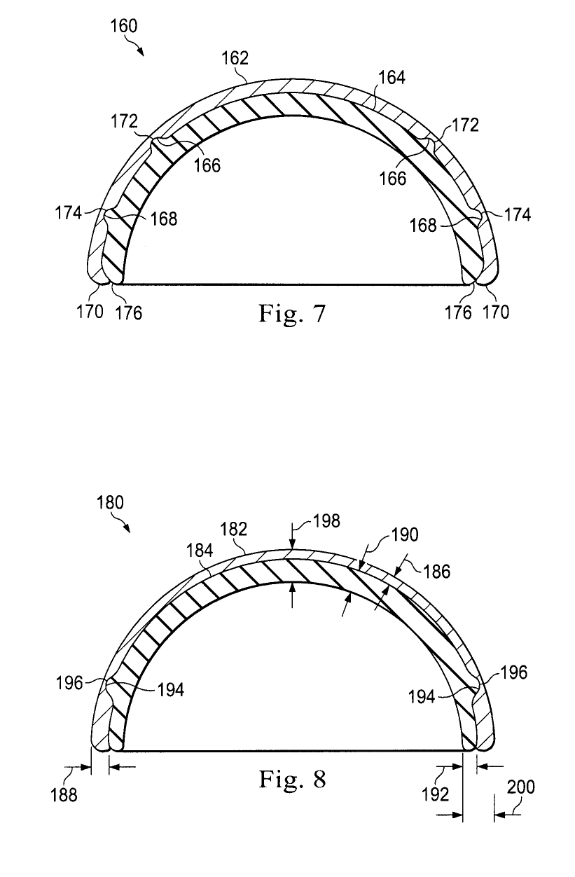

Referring now to FIG. 7, shown therein is a diagrammatic cross-sectional view of a prosthetic device 160 according to an alternative embodiment of the present disclosure. The prosthetic device 160 includes a outer shell component 162 and an inner articulating component 164. In some aspects the outer shell component 162 is similar to the shell 106 described above and, similarly, in some aspects the inner articulating component 164 is similar to the articulating component 108 described above. However, in the present embodiment, the outer shell component 162 includes a pair of continuous annular recesses 166 and 168 extending circumferentially about the inner surface. The outer shell component 162 also includes a continuous annular protrusion 170 that at least partially defines the rim or boundary of the shell component as shown. In that regard, the protrusion 170 is substantially similar to the protrusion 122 of the shell 106 described above. The recesses 166 and 168 are spaced from one another and positioned between the protrusion 170 and an apex of the inner surface. In the present embodiment, the outer surface of the articulating component 164 includes a pair of protrusions 172, 174 sized and shaped to engage the recesses 166, 168 of the shell component 162, respectively. In that regard, the protrusions 172, 174 have rounded profiles that are substantially the inverse of the profile of the recesses 166, 168 of the shell component 162. In some instances, the protrusions 172, 174 and the recesses 166, 168 snap-fit together. Further, the outer surface of the articulating component 164 includes a taper 176 adjacent its outer edge or rim. In that regard, the taper 176 is sized and shaped to mate with the protrusion 170 of the shell component 162. In some instances, the taper 176 and the protrusion 170 snap-fit together similar to the protrusions 172, 174 and the protrusions 166, 168. In this manner, the taper 176 is substantially similar to the taper 138 described above.

Referring now to FIG. 8, shown therein is a diagrammatic cross-sectional view of a prosthetic device 180 according to another embodiment of the present disclosure. The prosthetic device 180 includes a outer component 182 and an inner component 184. In some aspects the outer component 182 is similar to the shell 106 and/or the shell component 162 described above and, similarly, in some aspects the inner component 164 is similar to the articulating component 108 and/or the articulating component 164 described above. In the present embodiment, the outer component 182 has a thickness 186 that is generally less than about 1.0 mm. The outer component 182 has a thickness 188 adjacent its rim or outer boundary that is greater than the thickness 186 and, in some instances, is between about 0.75 mm and about 2.0 mm. In the present embodiment, the inner component 184 has a thickness 190 that is generally less than about 6.0 mm. The inner component 184 has a thickness 192 adjacent its rim or outer boundary that is less than the thickness 190 and, in some instances, is between about 1.0 mm and about 5.25 mm. The outer component 182 includes an annular recess 194 extending circumferentially about its inner surface. Generally, the outer component 182 has the thickness 188 between the recess 194 and its rim and the thickness 186 between the recess 194 and the apex of the outer component. In that regard, the difference in thicknesses 186, 188 at least partially defines the recess 194 in some instances. The inner component 184 includes an annular protrusion 196 extending circumferentially about its outer surface. The annular protrusion 196 is sized, shaped, and positioned to engage the recess 194 of the outer component 182 to secure the inner component 184 to the outer component 182. Generally, the inner component 184 has the thickness 192 between the projection 196 and its rim and the thickness 190 between the projection 196 and the apex of the inner component. In that regard, the difference in thicknesses 190, 192 at least partially defines the projection 196 in some instances. In some instances, the increased thickness 188 of the outer component 182 mating with the reduced thickness 192 of the inner component 184 helps to secure the components together.

When assembled together the outer component 182 and the inner component 184 have a thickness 198 between the outer surface of the outer component and the inner surface of the inner component between the interface of the projection 196 and recess 194 and the apex of the components. When assembled together the outer component 182 and the inner component 184 have a thickness 200 between the outer surface of the outer component and the inner surface of the inner component between the interface of the projection 196 and recess 194 and the rim of the components. In that regard, the thickness 198 is substantially equal to the thickness 200 in the present embodiment. Thus, the difference between the thicknesses 186 and 188 of the outer component and the difference between the thicknesses 190 and 192 of the inner component are substantially equal. In other instances, the differences between the thickness 186, 188 and 190, 192 are not substantially equal such that the thicknesses 198, 200 of the assembled components is not equal.

Generally, the shell or outer components of the present disclosure are formed of a material that is more rigid than the material of the articulating or inner components. For example, in some embodiments the shell is formed of a medical grade metal suitable for implantation, including but not limited to stainless steel alloys, cobalt-chrome alloys, titanium alloys, nickel-titanium alloys, and other suitable metals. In other embodiments, the shell is formed of a composite material, including but not limited to polyetheretherketone (PEEK), carbon-reinforced PEEK, Dyneema, and other suitable composites.

In some instances, the prosthetic devices of the present disclosure are fiber reinforced, include one or more deformation control elements, or comprise a material or combination of materials particularly suited for positioning within an articulating joint. In some embodiments, the prosthetic devices are formed of materials or combinations of materials as described in U.S. patent application Ser. No. 10/497,897 titled “CUSHION BEARING IMPLANTS FOR LOAD BEARING APPLICATIONS” and U.S. patent application Ser. No. 12/100,090 titled “MANUFACTURING AND MATERIAL PROCESSING FOR PROSTHETIC DEVICES”, each hereby incorporated by reference in its entirety.

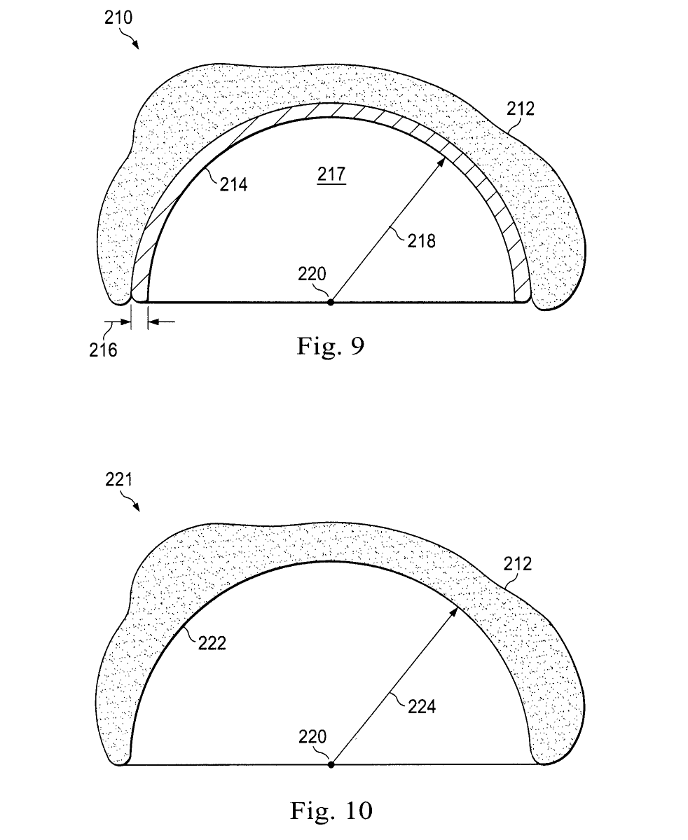

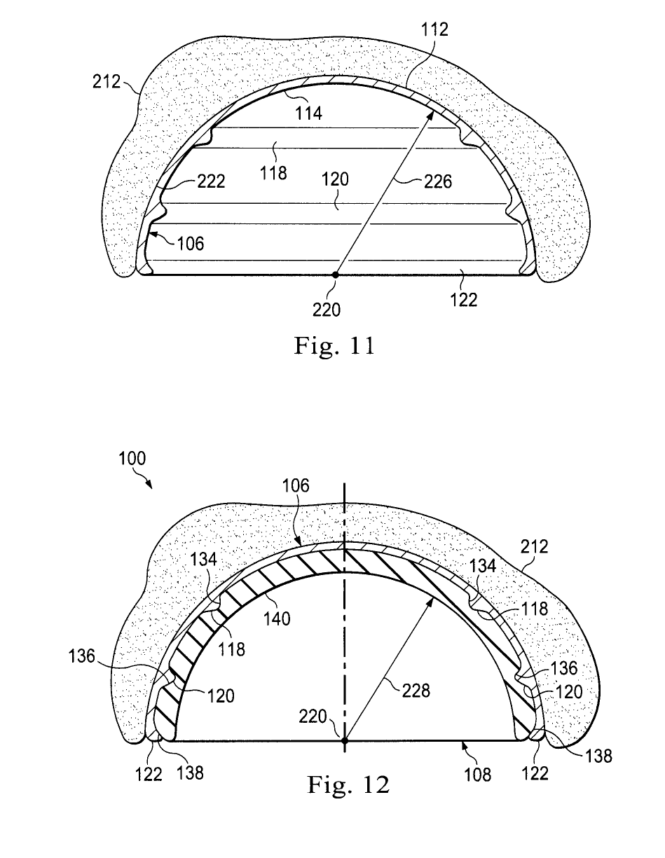

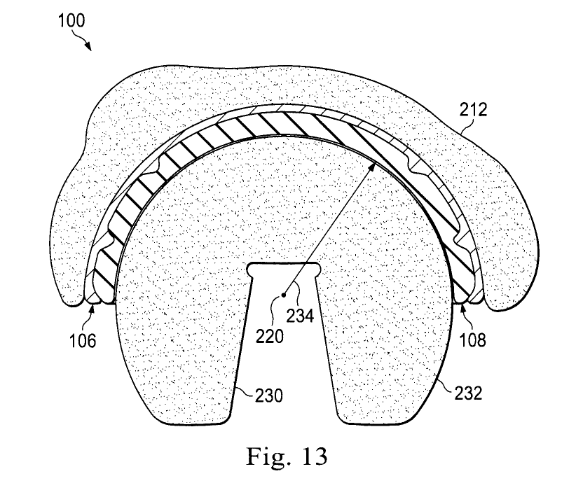

Referring now to FIGS. 9, 10, 11, 12, and 13, shown therein are various stages of the prosthetic device 104 described above being implanted. Specifically, FIG. 9 is a diagrammatic cross-sectional view of a patient's unprepared acetabulum 210; FIG. 10 is a diagrammatic cross-sectional view of a patient's prepared acetabulum 211; FIG. 11 is a diagrammatic cross-sectional view of the shell 106 implanted into the patient's prepared acetabulum 211; FIG. 12 is a diagrammatic cross-sectional view of the articulating component 108 assembled with the shell 106 and implanted into the patient's prepared acetabulum 211; and FIG. 13 is a diagrammatic cross-sectional view of the implanted articulating component 108 assembled with the shell 106 and mated with a femoral component.

Referring more specifically to FIG. 9, the patient's unprepared acetabulum 210 comprises a bony portion 212 and an articular cartilage portion 214. The articular cartilage 214 is shown as having a substantially uniform thickness 216. Generally, the thickness 216 of the articular cartilage 214 of a healthy acetabulum is approximately 4 mm or less. In the present embodiment the outer articulating surface 217 of the articular cartilage 214 is shown as being defined by a radius of curvature 218 extending from a center point 220. In some instances, the radius of curvature 218 of the articulating surface 217 is between about 16 mm and about 24 mm. However, the radius of curvature 218 is outside of this range for some patients. While the articular cartilage 214 is shown as having a substantially uniform thickness 216, it is recognized that the thickness of the articular cartilage will vary slightly to substantially across the acetabulum 210. For example, in some instances the articular cartilage 214 has degenerated, has been damaged, and/or has been at least partially removed. Accordingly, while the articular cartilage 214 is shown and at times discussed as having a substantially uniform thickness 216 it is understood that the present disclosure is equally applicable to articular cartilage 214 with a non-uniform thickness.

Referring more specifically to FIG. 10, preparation of the acetabulum includes reaming at least the articular cartilage 214 to define a surface 222 having a radius of curvature 224 for receiving the shell 106. In some instances, the surface 222 is partially spherical. That is, the surface 222 is defined as a portion of an outer surface of a sphere. In some particular embodiments, the surface 222 is substantially semi-spherical. In some instances, at least a portion of the bony portion 212 is reamed or cut to create the surface 222. In that regard, removing at least a portion of the bony portion 212 can help stimulate bone ingrowth between the surface 222 and the shell 106 after implantation of the shell. Generally, the radius of curvature 224 of the surface 222 is sized to match the radius of curvature 128 of the outer surface 112 of the shell 106.

Referring more specifically to FIG. 11, after the surface 222 has been prepared the shell 106 is implanted into the prepared acetabulum 211. In that regard, the shell 106 is fixedly secured to the surface 222 in some instances. Generally, the inner surface 114 of the shell 106 defines a radius of curvature 226 relative to center point 220, as shown. Referring more specifically to FIG. 12, once the shell 106 is secured within the prepared acetabulum 211 the articulating component 108 is positioned within the shell 106 such that the engagement of the projections 118, 120, 122 with the recesses 134, 136 and taper 138 anchors the articulating component 108 to the shell 106. The inner articulating surface 140 of the articulating component 108 has a radius of curvature 228 relative to the center point 220 after implantation. In some instances, the radius of curvature 228 is substantially similar to the radius of curvature 218 of the natural articular cartilage 214 prior to preparation of the acetabulum. In other instances, the radius of curvature 228 is less than the radius of curvature 218 of the natural articular cartilage 214 prior to preparation of the acetabulum. In some instances, the radius of curvature 228 is larger than the radius of curvature 218 of the natural articular cartilage 214 prior to preparation of the acetabulum.

Referring more specifically to FIG. 13, a femoral head 230 has been mated with the inner articulating surface 140 of the articulating component 108 to provide a functioning hip joint. The femoral head 230 includes an outer articulating surface 232 having a radius of curvature 234 relative to center point 220. The radius of curvature 234 is substantially equal to or slightly less than the radius of curvature 228 of the inner articulating surface 140. In some instances, the femoral head 230 is a natural femoral head. In some instances, the femoral head is a natural femoral head that has been reshaped or resurfaced for mating with the articulating component 108. In other instances, the femoral head 230 is an artificial femoral head. In some embodiments, the femoral head 230 is larger than the natural femoral head of the patient.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure. Also, it will be fully appreciated that variations of the above-disclosed and other features and functions, or alternatives thereof, may be combined into other methods, systems, apparatus, or applications. Similarly, various presently unforeseen or unanticipated alternatives, modifications, and/or variations of the present disclosure subsequently made by those skilled in the art are also encompassed by the present disclosure and the following claims.

1. A prosthetic device for implantation into a hip joint comprising:
a first component comprising an outer surface for securely engaging a bony portion of an acetabulum and an opposing inner surface for receiving a second component, the outer surface having a generally semi-spherical profile, the inner surface having a generally semi-spherical profile concentric with the outer surface, the first component having a substantially uniform thickness less than about 1.0 mm between the outer surface and the inner surface, the first component further comprising at least one annular protrusion extending circumferentially from the inner surface with a height less than about 1.0 mm relative to the inner surface, the first component comprising a substantially rigid first material; and
the second component comprising an outer surface having a generally semi-spherical profile for engagement with the inner surface of the first component, the outer surface comprising at least one annular recess extending circumferentially into the outer surface with a depth less than about 1.0 mm, the at least one annular recess shaped to receive the at least one annular protrusion of the first component, the second component further comprising an inner surface for mating with a femoral head, the inner surface having a generally semi-spherical profile concentric with the outer surface, the second component having a substantially uniform thickness less than about 3.0 mm between the outer surface and the inner surface, the second component comprising a pliable second material.

2. The prosthetic device of claim 1, wherein the substantially rigid first material comprises a metal.

3. The prosthetic device of claim 2, wherein the pliable second material comprises a material having a non-linear half bell-shaped stress-strain curve.

4. The prosthetic device of claim 3, wherein the pliable second material comprises a polycarbonate polyurethane having a hardness of approximately 80 Shore A.

5. The prosthetic device of claim 1, wherein the first and second components have a total thickness between the outer surface of the first component and the inner surface of the second component that is approximately equal to a thickness of a native articular cartilage of the acetabulum adjacent the bony portion of the acetabulum.

6. The prosthetic device of claim 1, wherein the thickness of the second component is at least twice the thickness of the first component.

7. The prosthetic device of claim 1, wherein the outer surface of the first component is defined by a radius of curvature between about 48.0 mm and about 60.0 mm.

8. The prosthetic device of claim 7, wherein the inner surface of the second component is defined by a radius of curvature between about 45.0 mm and about 59.0 mm.

9. The prosthetic device of claim 8, further comprising a prosthetic femoral head for mating with the inner surface of the second component.

10. The prosthetic device of claim 9, wherein the prosthetic femoral head comprises a partially spherical portion having a radius of curvature between about 45.0 mm and about 59.0 mm.

11. The prosthetic device of claim 1, wherein the first component comprises at least two annular protrusion extending circumferentially from the inner surface and wherein the second component comprises at least two annular recesses extending circumferentially into the outer surface.

12. A prosthetic acetabular cup system, comprising:
a metal shell comprising an outer surface for securely engaging a prepared portion of an acetabulum, an opposing inner surface for receiving a pliable articulating component, and at least one first engagement feature associated with the inner surface, the metal shell having a thickness less than about 1.0 mm between the outer surface and the inner surface; and
the pliable articulating component comprising an outer surface for engaging the inner surface of the metal shell including at least one second engagement feature associated with the outer surface and sized and shaped to engage the at least one first engagement feature of the metal shell, the pliable articulating component further comprising an inner surface for receiving a femoral head, the pliable articulating component having a thickness less than about 3.0 mm between the outer surface and the inner surface.

13. The system of claim 12, wherein the at least one first engagement feature comprises at least one recess and the at least one second engagement feature comprises at least one projection.

14. The system of claim 12, wherein the at least one first engagement feature comprises at least one projection and the at least one second engagement feature comprises at least one recess.

15. The prosthetic device of claim 12, wherein the metal shell and the pliable articulating component have a total thickness between the outer surface of the metal shell and the inner surface of the pliable articulating component that is approximately equal to a thickness of a native articular cartilage of the acetabulum.

16. The prosthetic device of claim 12, wherein the thickness of the second component is at least twice the thickness of the first component.

17. The prosthetic device of claim 1, wherein the outer surface of the first component is defined by a radius of curvature between about 24.0 mm and about 30.0 mm and the inner surface of the second component is defined by a radius of curvature between about 21.0 mm and about 28.5 mm.

18. A method of implanting an artificial acetabular component comprising:
removing at least a portion of an articular cartilage of an acetabulum to access a bony portion of the acetabulum;
engaging an outer surface of a metal shell with the bony portion of the acetabulum, the metal shell having an opposing inner surface and a thickness of less than about 1.0 mm between the outer surface and the inner surface;
snap-fitting a pliable articulating component into engagement with the inner surface of the metal shell, the pliable articulating component having a thickness less than about 3.0 mm between an outer engagement surface and an inner articulating surface such that when snap-fittingly engaged the metal shell and the pliable articulating component have a total thickness between the outer surface of the metal shell and the inner articulating surface of the pliable articulating component that is approximately equal to a thickness of the removed articular.

19. The method of claim 18, wherein snap-fitting the pliable articulating component into engagement with the inner surface of the metal shell comprises snap fitting at least one projection extending from the inner surface of the metal shell with at least one recess extending into the outer engagement surface of the pliable articulating component.

20. The method of claim 19, further comprising wetting at least the outer engagement surface of the pliable articulating component prior to snap-fitting the pliable articulating component into engagement with the metal shell.
